SEQUENCE LISTING (3 sequences)

SEQ ID NO: 1 - protein, 7 aa, synthetic construct
WLKPDPS

SEQ ID NO: 2 - protein, 10 aa, synthetic construct
NESPEQTDDL

SEQ ID NO: 3 - protein, 6 aa, synthetic construct
WLKPDP

(19) United States
(12) Patent Application Publication
(10) Pub. No.: US 2026/0103487 A1
(43) Pub. Date: Apr. 16, 2026

(54) LACTOPONTIN-DERIVED POLYPEPTIDE THAT PROMOTES OSTEOBLAST PROLIFERATION AND DIFFERENTIATION AND APPLICATION THEREOF

(71) Applicants: Jiangnan University, Wuxi (CN); Le Bonta Wellness Co., Ltd, Shanghai (CN)

(72) Inventors: Peng Zhou, Wuxi (CN); Yipin Lyu, Wuxi (CN); Jie Zhang, Wuxi (CN); Xinyu Ge, Wuxi (CN); Dasong Liu, Wuxi (CN); Changshu Liu, Shanghai (CN); Kexin Li, Shanghai (CN)

(21) Appl. No.: 19/419,234

(22) Filed: Dec. 15, 2025

Related U.S. Application Data
(63) Continuation of application No. PCT/CN2025/122907, filed on Sep. 22, 2025.

(30) Foreign Application Priority Data
Apr. 15, 2025 (CN) 2025104706375

(51) Int. Cl. C07K 7/06; A61K 9/00; A61K 9/06; A61K 38/00; A61K 47/10; A61K 47/24; A61K 47/36; A61K 47/42; A61K 47/44; A61P 21/00

(52) U.S. Cl. CPC C07K 7/06; A61K 9/0056; A61K 9/06; A61K 47/10; A61K 47/24; A61K 47/36; A61K 47/42; A61K 47/44; A61P 21/00; A61K 38/00

(57) ABSTRACT

This disclosure relates to the field of neurology. More specifically, disclosed herein are several osteopontin mimetic peptides that are useful for the treatment of stroke and other ischemic injuries. Also disclosed are methods for the treatment of cellular damage due to excitotoxic injury, ischemia, and/or hypoxia by administering an osteopontin peptide mimetic.

### REFERENCE TO SEQUENCE LISTING

The instant application contains a Sequence Listing in XML format as a file named “PC250019A.xml”, created on 2025 Nov. 25, of 3,835 byte in size, and which is hereby incorporated by reference in its entirety.

### TECHNICAL FIELD

The present disclosure relates to a lactopontin-derived polypeptide that promotes osteoblast proliferation and differentiation and an application thereof, and belongs to the technical field of bioactive peptides.

### BACKGROUND

Bone mineral density (BMD) mainly refers to a density of bone mineral content per unit volume of bone, which primarily reflects the toughness of bones. Improving BMD is an important measure for maintaining bone health, and especially has a significant effect on preventing osteoporosis. Clinically, chemical drugs are still dominant for treating osteoporosis, but there usually exist certain risks, and for example, long-term use may cause gastrointestinal discomfort, cardiovascular diseases, and other illnesses. Therefore, there is increasing interest in finding safer, food-derived natural substitutes that promote bone formation and reverse bone structure damage.

Polypeptides are protein degradation products, and various bioactive peptides have been reported to have functions of regulating immunity, preventing cardiovascular diseases, and promoting bone health. These peptides may be obtained through microbial fermentation, digestive enzymatic hydrolysis, genetic engineering-based artificial synthesis, chemical synthesis, and other means. Most bioactive peptides are composed of 2-20 amino acid residues and are easily digested and absorbed by the human body. There are abundant types of milk-derived polypeptides, but there are fewer studies related to bone health. In 2007, Huttunen et al. reported that milk-derived tripeptides IPP have the effect of promoting osteoblast proliferation, differentiation, and signal transduction. However, compared with those of the milk-derived polypeptides of rich variety, research reports thereon are still relatively scarce. Lactopontin (LPN) is an important active protein from milk of mammals, and LPN promotes bone development in growing mice, but it remains unclear whether osteogenically active peptides that regulate bone health are produced after gastrointestinal digestion.

Therefore, it is of great significance to explore LPN-derived bioactive peptides that promote bone health.

### SUMMARY

To solve the above problems, the present disclosure provides a milk-derived bioactive peptide WLKPDPS or NE(pS)PEQTDDL, and the bioactive peptide exhibits extremely high biological safety and has functions of promoting osteoblast proliferation and differentiation.

A first objective of the present disclosure is to provide a milk-derived bioactive peptide, and an amino acid sequence of the milk-derived bioactive peptide is WLKPDPS (SEQ ID NO: 1) or NESPEQTDDL (SEQ ID NO: 2).

In an embodiment, serine in the milk-derived bioactive peptide NESPEQTDDL may be phosphorylated serine.

In an embodiment, the milk-derived bioactive peptide is WLKPDPS or NE(pS)PEQTDDL.

In an embodiment, a method for preparing the milk-derived bioactive peptide include a solid-phase synthesis method, an enzymatic hydrolysis method, and a microbial expression method.

A second objective of the present disclosure is to provide a food, drug, health product, or nutritional product, and the food, drug, health product, or nutritional product contains an effective dose of any of the above milk-derived bioactive peptides; and

optionally, the food, drug, health product, or nutritional product may also contain any of the above milk-derived bioactive peptides; a derivative of the milk-derived bioactive peptide refers to a milk-derived bioactive peptide derivative obtained by performing hydroxylation, carbonylation, carboxylation, methylation, acetylation, phosphorylation, esterification, or glycosylation modification on an amino acid side chain group, amino terminus, or carbonyl terminus of the milk-derived bioactive peptide.

In an embodiment, the drug further contains a pharmaceutically acceptable excipient; and the pharmaceutically acceptable excipient refers to a conventional pharmaceutical carrier in the pharmaceutical field.

In an embodiment, the excipient includes one or more of the following: a binder such as a cellulose derivative, alginate, gelatin, or polyvinylpyrrolidone; a diluent such as starch, pregelatinized starch, dextrin, sucrose, lactose, or mannitol; a filler such as starch or sucrose; a humectant such as glycerol; a disintegrant such as sodium carboxymethyl starch, cross-linked polyvinylpyrrolidone, or dry starch; an absorption enhancer such as a quaternary ammonium compound; a surfactant such as polysorbate, sorbitan fatty acid ester, or fatty acid glyceride; a colorant such as titanium dioxide, sunset yellow, methylene blue, or pharmaceutical-grade iron oxide red; a lubricant such as hydrogenated vegetable oil, talc, or polyethylene glycol; a coating material such as acrylic resin, hydroxypropyl methylcellulose, povidone, or cellulose acetate phthalate; and any other adjuvant such as a flavoring agent or sweetener added to a composition;
optionally, a dosage form of the drug includes but is not limited to an oral dosage form, an injectable dosage form, and an inhalation dosage form;
optionally, the oral dosage form includes but is not limited to a tablet, a capsule, a granule, an oral liquid, and an oral suspension;
optionally, the injectable dosage form includes but is not limited to an injection and an injection powder; and
optionally, the inhalation dosage form includes but is not limited to an aerosol and a dry powder inhaler.

In an embodiment, the food includes but is not limited to a cereal product, a vegetable product, a fruit product, a meat product, a seafood, an egg product, a dairy product, a soybean product, and a beverage;
the food further includes a special dietary food; and
the health product further contains an acceptable excipient.

In an embodiment, the health product includes a gel candy.

In an embodiment, the gel candy is a soft candy, and is prepared by the following steps:
(1) adding gelatin, starch, oligosaccharides, edible glycerol, and water to a mixing barrel, stirring for 20-40 minutes, and decocting to obtain a gel solution;
(2) adding WLKPDPS or NE(pS)PEQTDDL polypeptide dry powder, fruit flavor, phospholipid, and sunflower seed oil to the mixing barrel, and stirring for 30 minutes to obtain a filling solution;
where a mass ratio of gelatin to starch to oligosaccharides to edible glycerol to water to polypeptide dry powder to fruit flavor to phospholipid and to sunflower seed oil is 2-6:0.5-2:0.5-2:8-12:4-8:0.1-1:0.1-0.5:0.3-0.8:5-10; and
(3) molding the gel solution and the filling solution through a candy machine, and cooling at 20-25° C. for 60-80 minutes to obtain a polypeptide gel candy.

A third objective of the present disclosure is to provide an application of any of the above milk-derived bioactive peptides in the preparation of a food, drug, health product, or nutritional product, where the food, drug, health product, or nutritional product is used for promoting calcium absorption.

In an embodiment, the milk-derived bioactive peptide may also be used for preparing products with functions of antioxidation, anti-inflammation, cartilage repair and tissue regeneration (e.g., promoting bone matrix mineralization, enhancing bone mineral density, accelerating bone tissue growth, and shortening fracture healing cycles), metabolic regulation and immunization (e.g., regulating enzyme activity and enhancing immunity), prevention of dental caries, and digestive function improvement.

In an embodiment, the food includes but is not limited to a cereal product, a vegetable product, a fruit product, a meat product, a seafood, an egg product, a dairy product, a soybean product, and a beverage;
the food further includes a special dietary food; and
the health product further contains an acceptable excipient.

In an embodiment, the food, drug, health product, or nutritional product is used for promoting calcium absorption.

In an embodiment, the drug further contains a pharmaceutically acceptable excipient; and the pharmaceutically acceptable excipient refers to a conventional pharmaceutical carrier in the pharmaceutical field.

In an embodiment, the excipient includes one or more of the following: a binder such as a cellulose derivative, alginate, gelatin, or polyvinylpyrrolidone; a diluent such as starch, pregelatinized starch, dextrin, sucrose, lactose, or mannitol; a filler such as starch or sucrose; a humectant such as glycerol; a disintegrant such as sodium carboxymethyl starch, cross-linked polyvinylpyrrolidone, or dry starch; an absorption enhancer such as a quaternary ammonium compound; a surfactant such as polysorbate, sorbitan fatty acid ester, or fatty acid glyceride; a colorant such as titanium dioxide, sunset yellow, methylene blue, or pharmaceutical-grade iron oxide red; a lubricant such as hydrogenated vegetable oil, talc, or polyethylene glycol; a coating material such as acrylic resin, hydroxypropyl methylcellulose, povidone, or cellulose acetate phthalate; and any other adjuvant such as a flavoring agent or sweetener added to a composition;
optionally, a dosage form of the drug includes but is not limited to an oral dosage form, an injectable dosage form, and an inhalation dosage form;
optionally, the oral dosage form includes but is not limited to a tablet, a capsule, a granule, an oral liquid, and an oral suspension;
optionally, the injectable dosage form includes but is not limited to an injection and an injection powder; and
optionally, the inhalation dosage form includes but is not limited to an aerosol and a dry powder inhaler; and
optionally, the food includes but is not limited to a cereal product, a vegetable product, a fruit product, a meat product, a seafood, an egg product, a dairy product, a soybean product, and a beverage; the food further includes a special dietary food; and the health product further contains an acceptable excipient.

In an embodiment, the health product includes a gel candy.

The present disclosure has the following beneficial effects:

The milk-derived bioactive polypeptides WLKPDPS and NE (PS) PEQTDDL of the present disclosure have a significant effect of promoting osteoblast proliferation and osteoblast mineralization, and are expected to be used for the preparation of drugs for promoting osteoblast proliferation and preventing osteoporosis.

The milk-derived bioactive polypeptides WLKPDPS and NE (PS) PEQTDDL of the present disclosure are synthesized conveniently and may be industrially produced, having broad application prospects in the fields of foods, drugs, cosmetics, and the like.

### BRIEF DESCRIPTION OF THE DRAWINGS

FIG. 1 illustrates HPLC detection results of a milk-derived bioactive peptide WLKPDPS.

FIG. 2 illustrates LC-MS detection results of a milk-derived bioactive peptide WLKPDPS.

FIG. 3 illustrates HPLC detection results of a milk-derived bioactive peptide NE(pS)PEQTDDL.

FIG. 4 illustrates LC-MS detection results of a milk-derived bioactive peptide NE(pS)PEQTDDL.

FIG. 5 illustrates effects of milk-derived bioactive peptides WLKPDPS and NE(pS)PEQTDDL on osteoblast proliferation.

FIG. 6 illustrates effects of milk-derived bioactive peptides WLKPDPS and NE(pS)PEQTDDL on osteoblast differentiation.

FIG. 7 illustrates effects of milk-derived bioactive peptides WLKPDPS and NE(pS)PEQTDDL on osteoblast-related pathways.

FIG. 8A illustrates effects of a milk-derived bioactive peptide WLKPDPS on body weight of rats.

FIG. 8B illustrates effects of a milk-derived bioactive peptide WLKPDPS on terminal body weight of rats.

FIG. 8C illustrates effects of a milk-derived bioactive peptide WLKPDPS on body length of rats.

FIG. 9A illustrates effects of a milk-derived bioactive peptide WLKPDPS on femoral length of rat femurs.

FIG. 9B illustrates effects of a milk-derived bioactive peptide WLKPDPS on femoral wet weight of rat femurs.

FIG. 9C illustrates effects of a milk-derived bioactive peptide WLKPDPS on femoral dry weight of rat femurs.

FIG. 9D illustrates effects of a milk-derived bioactive peptide WLKPDPS on femoral width of rat femurs.

FIG. 9E illustrates effects of a milk-derived bioactive peptide WLKPDPS on femoral thickness of rat femurs.

FIG. 10A illustrates effects of a milk-derived bioactive peptide WLKPDPS on maximum load of rat femurs.

FIG. 10B illustrates effects of a milk-derived bioactive peptide WLKPDPS on maximum deflection of rat femurs.

FIG. 11 illustrates Micro-CT three-dimensional reconstruction results of rat femurs treated with a milk-derived bioactive peptide WLKPDPS.

FIG. 12A illustrates effects of a milk-derived bioactive peptide WLKPDPS on bone density of rat bones.

FIG. 12B illustrates effects of a milk-derived bioactive peptide WLKPDPS on bone volume fraction of rat bones.

FIG. 12C illustrates effects of a milk-derived bioactive peptide WLKPDPS on trabecular thickness of rat bones.

FIG. 12D illustrates effects of a milk-derived bioactive peptide WLKPDPS on trabecular number of rat bones.

FIG. 12E illustrates effects of a milk-derived bioactive peptide WLKPDPS on trabecular separation of rat bones.

### DETAILED DESCRIPTIONS OF THE EMBODIMENTS

Preferred embodiments of the present disclosure are described below, and it should be understood that the embodiments are intended to better explain the present disclosure and are not intended to limit the present disclosure.

### Raw Materials Used in the Examples:

Lactopontin (LPN) was isolated from fresh cow milk, and a specific method is referenced in the literature (Ma Ping, Sun Jie, Liu Ning. Isolation, purification and identification of osteopontin from cow milk [J]. Food and Fermentation Industries, 2008, (06): 135-139); and
Pepsin, pancreatin, and bile salts were purchased from Sigma-Aldrich, USA; and potassium chloride, sodium chloride, and sodium hydroxide were purchased from Sinopharm Chemical Reagent Co., Ltd.

Milk-derived bioactive peptides used in the examples were all synthesized by Shanghai Science Peptide Biological Technology Co., Ltd upon commission.

### Test Methods:

### 1. HPLC Method

High-performance liquid chromatography (HPLC) is adopted, with a Kromasil 100-5-C18 (4.6×250 mm×5 μm) chromatographic column, a 0.1% trifluoroacetic acid aqueous solution (A) and a 0.1% trifluoroacetic acid acetonitrile solution (B) as mobile phases, a total flow rate of 1 mL/minute, and a detection wavelength set to 214 nm. Samples are dissolved in ultrapure water, and an injection volume is 7 μL. A gradient elution program is as follows:

| Time (min) | Phase A (%) | Phase B (%) |
|---|---|---|
| 0.01 | 90 | 10 |
| 30 | 60 | 40 |
| 33 | 0 | 100 |
| 38 | 0 | 100 |
| 40 | 0 | 10 |
| 50 | End | |

### 2. LC-MS Method

An electrospray ionization (ESI) interface is used, with a detector voltage set to −0.2 kV, a CDL temperature of 250° C., a CDL voltage of 0 V, a blocking temperature of 200° C., a nebulizing gas flow rate of 1.5 L/minute, a preset bias of +4.5 kV, and T-Flow of 0.2 mL/min. The samples are dissolved in a solvent mixture composed of 15% acetonitrile (ACN) and 85% water (H₂O), with an injection volume of 0.2 μL.

### Example 1: Preparation of Milk-Derived Bioactive Peptides

Milk-derived bioactive peptides were prepared by using an infant model for in vitro simulated digestion, with the steps as follows:

### (1) Gastric Digestion Stage:

10 mg/mL of an LPN solution was added to an enzyme reactor for preheating with a pH value adjusted to 5.3, and mixed with simulated gastric fluid (pH 5.3, 724.3 U pepsin/mL, 13 mM potassium chloride, and 94 mM sodium chloride) at a ratio of 63:37 (v/v); after a reaction at 37° C. and pH 5.3 for 60 minutes, the pH value was adjusted to 7.0 with 2 M sodium hydroxide to terminate the digestion at this stage and obtain a gastric digestion product;

### (2) Intestinal Digestion Stage:

a pH value of the gastric digestion product was adjusted to 6.6, and mixing with simulated intestinal fluid (pH 6.6, 42.1 U pancreatin/mL, 8.2 mM bile salts, 10 mM potassium chloride, and 249 mM sodium chloride) was performed at a ratio of 62:38 (v/v); and a resulting mixture was digested at 37° C. and pH 6.6 for 60 minutes, and the intestinal digestion was terminated at 95° C. for 10 minutes, to obtain a final digestion product; and

### (3) Identification of Polypeptide Composition:

the final digestion product was analyzed via LC-MS/MS equipped with an online nano-electrospray ionization source. The entire system was an Orbitrap Fusion Lumos mass spectrometer (Thermo Fisher Scientific, MA, USA) coupled with EASY-nanoLC 1200. A 5 μL sample was loaded (an analytical column: Acclaim PepMap C18, 75 μm×25 cm), the sample was separated with a 60-minute gradient at a column flow rate of 300 nL/minute, a column temperature controlled at 40° C., and an electrospray voltage of 2 kV, and a gradient initiated at 4% (phase B), increased nonlinearly to 50% within 53 minutes and 40 seconds, increased to 95% within 40 s, and maintained for 5 minutes and 40 seconds. The mass spectrometer operated in a data-dependent acquisition mode, automatically switching between MS acquisition and MS/MS acquisition.

Mass spectrometry parameters were set as follows: (1) MS: a scanning range (m/z): 100-1,500; a resolution: 120,000; Normalized AGC target: 200%; a maximum injection duration: 100 ms; (2) HCD-MS/MS: a resolution: 50,000; Normalized AGC target: 200%; maximum injection time: 86 ms; collision energy: 25%, 30%, and 35%; and dynamic exclusion time: 30 s. Tandem mass spectra were analyzed by PEAKS Studio version 10.6 (Bioinformatics Solutions Inc., Waterloo, Canada). A database was Uniprot-Bos taurus (version 2024, 26635 entries). Database search parameters included: a fragment ion mass tolerance: 0.02 Da, a parent ion mass tolerance: 10 ppm, a maximum number of missed cleavages: 2, fixed modification: Carbamidomethylation 57.02, variable modifications: Oxidation (M) 15.99, Deamidation (NQ) 0.98, Acetylation (Protein N-term) 42.01, Phosphorylation (STY) 79.97. Protein identifications were filtered at 1% false discovery rate (FDR) with at least one unique peptide; and peptide identifications were filtered at 1% FDR.

A total of 242 peptide segments derived from LPN were identified, molecular docking of polypeptides was performed against a target BMPRIA, and potential milk-derived bioactive peptides WLKPDPS and NE(pS)PEQTDDL were screened based on binding energy.

Milk-derived bioactive peptides WLKPDP (SEQ ID NO: 3), WLKPDPS (SEQ ID NO: 1), and NE(pS)PEQTDDL were synthesized by Shanghai Science Peptide Biological Technology Co., Ltd. upon commission.

### Example 2: Detection of Milk-Derived Bioactive Peptides

Detection results of the milk-derived bioactive peptides WLKPDPS and NE(pS)PEQTDDL prepared in Example 1 are as follows:

### (1) HPLC Detection Results

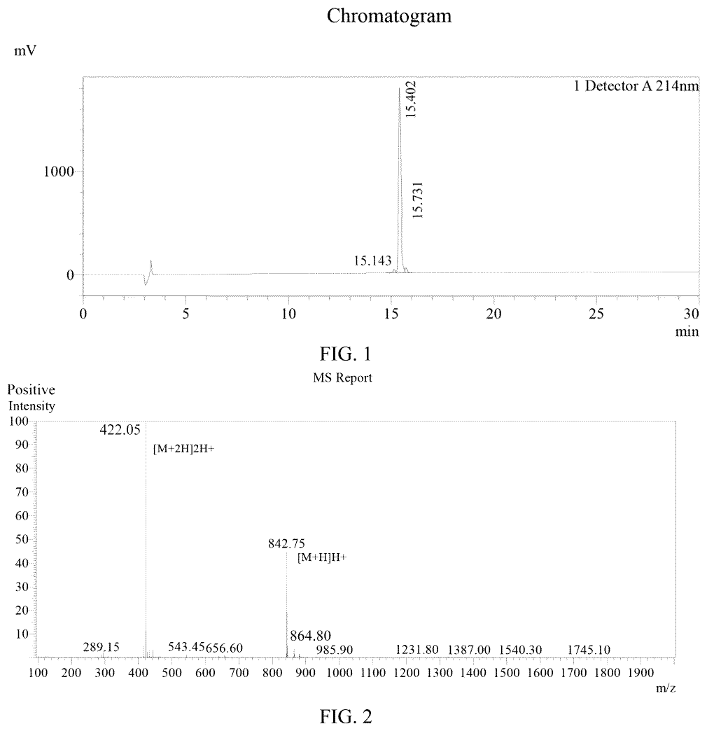

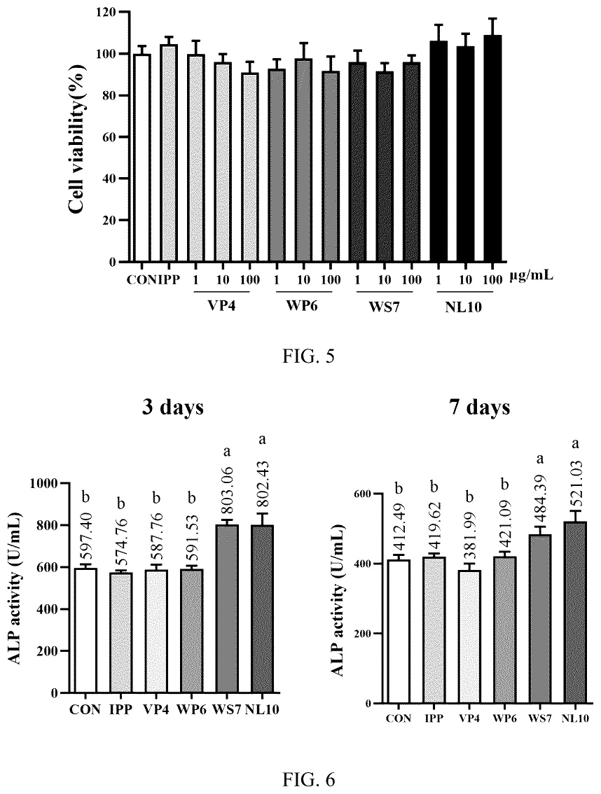

The HPLC detection results are shown in FIG. 1 (WLKPDPS) and FIG. 3 (NE(pS)PEQTDDL).

### (2) LC-MS Detection Results

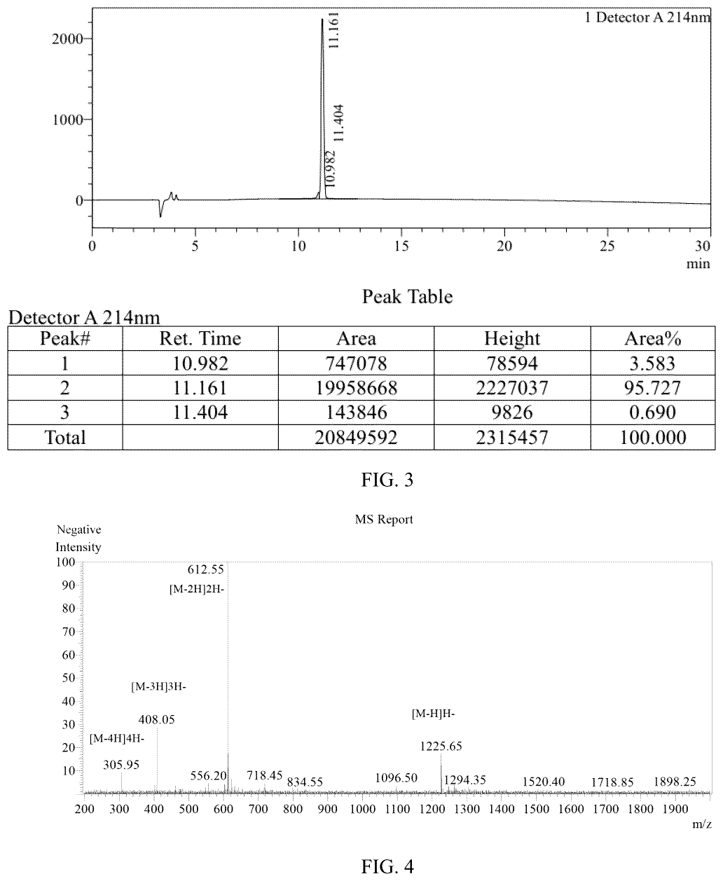

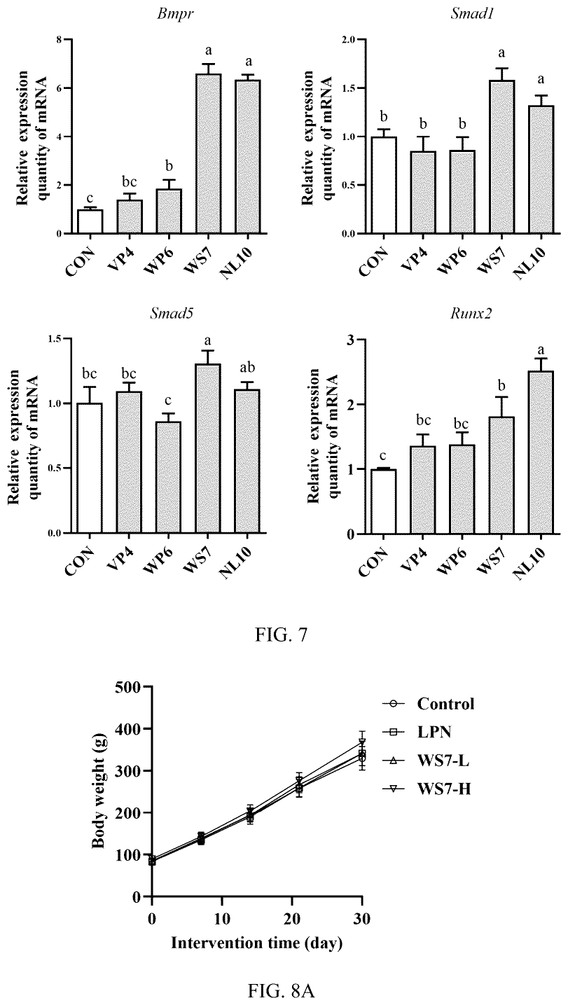

The LC-MS detection results are shown in FIG. 2 (WLKPDPS) and FIG. 4 (NE(pS)PEQTDDL).

### (3) Osteoblast Promotion Effect

Effects of the milk-derived bioactive peptides VFTP (VP4), WLKPDP (WP6), WLKPDPS (WS7), and NE(pS)PEQTDDL (NL10) on osteoblast proliferation were detected by the following steps:

MC3T3-E1 Subclone14 cells were seeded at a density of 5,000 cells/well in a 96-well plate. After incubation for 24 hours, samples were dissolved in α-MEM medium and sterilized by filtration through a 0.22 μm membrane, media containing 100 μg/mL IPP, 1, 10, 100 μg/mL VP4, WP6, WS7, and NL10 were added to the well plate respectively, and after incubation for 24 hours, a CCK-8 reagent was used to detect the number of cells.

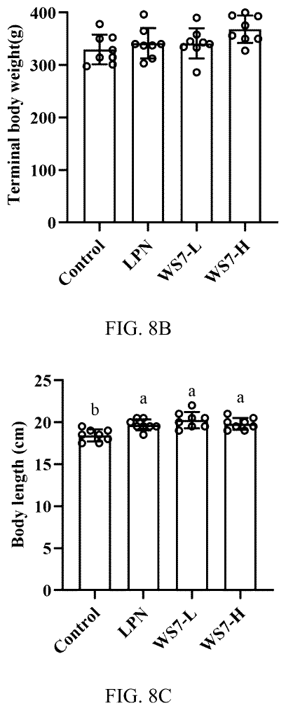

The results are shown in FIG. 5 and indicate that none of all the samples has significant effect on cell viability, suggesting that 100 μg/mL IPP, 1, 10, 100 μg/mL VP4, WP6, WS7, and NL10 have no cytotoxicity to MC3T3-E1 Subclone14 cells.

### (4) Effects on Osteoblast Differentiation

The effect of the milk-derived bioactive peptide WLKPDPS on osteoblast differentiation was detected by the following steps:

Cells were seeded in a 12-well plate at a density of 1×10⁵ cells/well, and 1 mL of a medium was added to each well. After 24 hours, the medium was replaced with an induction medium containing 100 μg/mL IPP, 100 μg/mL VP4, WP6, WS7, and NL10 respectively, and the medium was changed every two days. After 3 days and 7 days of intervention, a cell culture supernatant was collected, and ALP activity of the supernatant was determined by using an ALP kit.

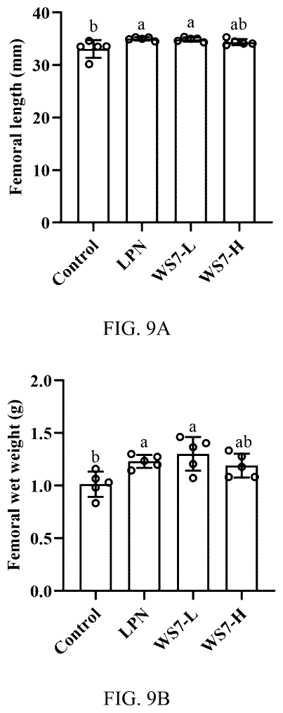

The results are shown in FIG. 6 and indicate that after 3 days (left panel) and 7 days (right panel) of induced differentiation, compared with a control group and an IPP group, WLKPDPS significantly enhanced the activity of alkaline phosphatase in the cell culture supernatant (different lowercase letters indicate significant differences between groups, p<0.05); and on day 3, the ALP activity of a WS7 group was 803.06±22.19 U/mL, and that of a NL10 group was 802.43±53.39 U/mL, which were 39.7% and 39.6% higher than that of the IPP group respectively, while similar results were observed on day 7.

### (5) Effects on Osteoblast-Related Pathways

The effect of the milk-derived bioactive peptide WLKPDPS on osteoblast differentiation was detected by the following steps:

Cells were seeded in a 12-well plate at a density of 1×10⁵ cells/well, and 1 mL of a medium was added to each well. After 24 hours, the medium was replaced with an induction medium containing 100 μg/mL WLKPDPS, and the medium was replaced every two days; and after 3 days of intervention, cellular RNA was extracted using an animal RNA extraction kit and reverse-transcribed to cDNA, and expression levels of genes related to a BMP/Smad pathway were determined by QPCR.

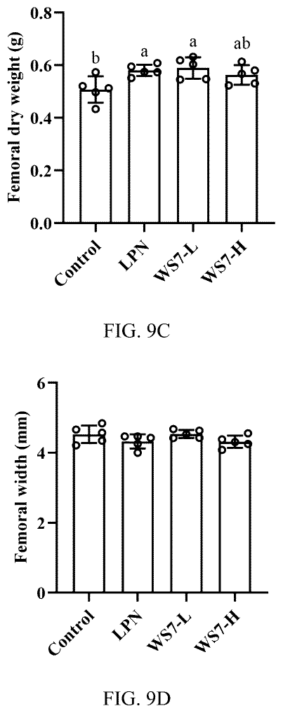

The results are shown in FIG. 7 and indicate that compared with the control group, WLKPDPS significantly upregulate mRNA expression levels of Bmpr, Smad1, Smad5, and Runx2 by 558.9%, 58.2%, 30.1%, and 81.4% respectively; and NE(pS)PEQTDDL significantly upregulate mRNA expression levels of Bmpr, Smad1, Smad5, and Runx2 by 533.9%, 32.3%, 10.6%, and 151.8% respectively.

### Example 3: Effects of Milk-Derived Bioactive Peptides on Bone Growth and Development of Rats

A milk-derived bioactive peptide WLKPDPS prepared in Example 1 was used in a vivo animal experiment. Thirty-two 3-week-old male SPF-grade Sprague-Dawley (SD) rats were purchased from Vital River Laboratory Animal Technology Co., Ltd. The rats had free access to food and water, with an environmental temperature of 20° C.-26° C. and a relative humidity of 40%-70%.

After acclimation, the rats were randomly divided into four groups:
(1) a control group (Control, n=8), intragastrically administered with deionized water per day;
(2) an LPN group (LPN, n=8), intragastrically administered with 15 mg/kg·BW LPN per day;
(3) a low-dose WS7 group (WS7-L, n=8), intragastrically administered with 0.41 mg/kg·BW polypeptide WLKPDPS per day; and
(4) a high-dose WS7 group (WS7-H, n=8), intragastrically administered with 4.1 mg/kg·BW polypeptide WLKPDPS per day. Body weights of the rats were recorded weekly, and the experiment ended after intragastric administration for 4 consecutive weeks.

The effects of the milk-derived bioactive peptide on the rats were detected as follows:

### 1. Effects on Growth Performance of Rats

The effects of the milk-derived bioactive peptide WLKPDPS on the growth performance of rats were detected by the following steps: After the rats were anesthetized with isoflurane, a body length of each of the rats, namely a distance from a nasal tip to a tail base, was measured with a straight ruler.

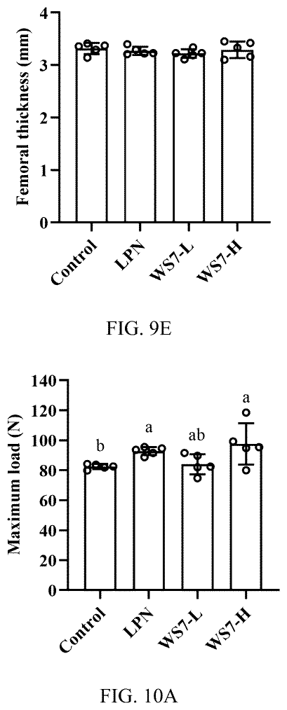

According to the results shown in FIG. 8A˜FIG. 8C, growth curves of the rats in each group exhibited high consistency during an experimental cycle, and there were no significant differences in final body weights of the rats in each group (p>0.05), and an average body weight of the rats in the high-dose WS7 group was the highest, which was 9.83% higher than that of the control group. After intervention with LPN and low-dose and high-dose WS7, body lengths of the rats increased by 6.78%, 9.83%, and 7.45% respectively (p<0.05), and there were no significant differences in the growth-promoting effect between intervention groups (p>0.05).

### 2. Effects on Apparent Indicators of Rat Femurs

The effects of the milk-derived bioactive peptide WLKPDPS on the apparent indicators of rat femurs were detected by the following steps: The rat femurs were isolated, muscles on bones were removed, and femoral parameters were measured with a vernier caliper.

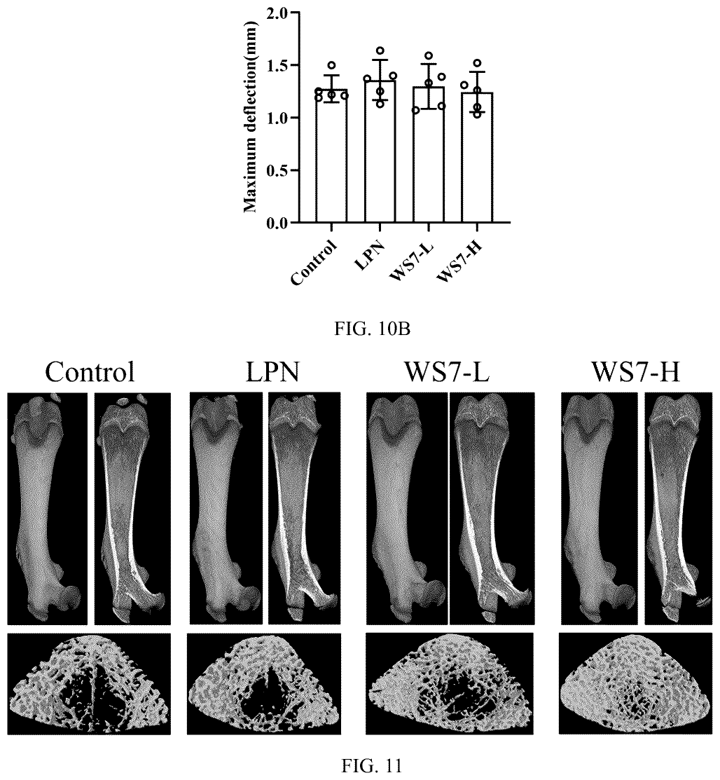

According to results shown in FIG. 9A˜FIG. 9E, compared with those of the control group, an average femoral length, wet weight, and dry weight of the rats in the LPN intervention group significantly increased by 5.84%, 21.31%, and 14.33% (p<0.05), while the low-dose WS7 induced an equivalent bone growth-promoting effect: increase amplitudes of this group in length (+5.45%, p<0.05), wet weight (+28.27%, p<0.05), and dry weight (+16.09%, p<0.05) were not statistically different from those of the LPN group (p>0.05). Although the high-dose WS7 (WS7-H) showed a positive trend, no significant difference (p>0.05) was found, indicating a dose threshold effect. There was no impact on femoral width or thickness in any treatment group (p>0.05).

### 3. Effects on Mechanical Properties of Rat Bones

The effects of the milk-derived bioactive peptide WLKPDPS on the mechanical properties of rat bones were detected by the following steps: A TA.XT plus analyzer was used to perform a three-point bending test on femurs of the rats, with a 12 mm support span (an HDP/3 PB probe) and a crosshead speed of 0.6 mm/min. The bone was always oriented to apply load to a femoral midshaft region. A load-displacement curve of each femur was used to determine an elastic load, a maximum load (N), and a displacement at the maximum load, i.e., a maximum deflection (mm).

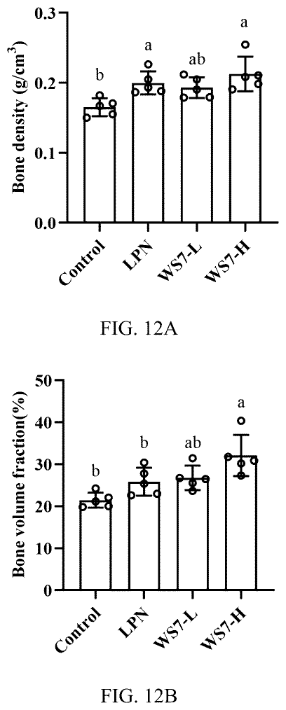

According to the results shown in FIG. 10A˜FIG. 10B, both LPN and WS7-H interventions significantly increased maximum femoral loads (p<0.05), which were 12.57% and 18.43% higher than that of the control group respectively, while WS7-L did not exhibit a significant promoting effect (p>0.05); and in terms of bone toughness, there was no significant difference between the groups in the maximum deflection (p>0.05). This indicates that WS7 reproduced an improvement effect of LPN on bone strength only at a high dose.

### 4. Effects on a Microstructure of Rat Bones

The effects of the milk-derived bioactive peptide WLKPDPS on the microstructure of rat bones were detected by the following steps: Isolated femurs were placed and fixed in a 4% paraformaldehyde solution for 24-48 hours a specimen was extracted and rinsed with PBS three times, and then stored in a 75% ethanol solution at 4° C. for later use. The treated femurs were scanned by a small animal scanning imaging system Micro-CT (Bruker SkyScan 1276) under the following conditions: a resolution of 18 μm, a voltage of 65 kV, current of 385 μA, and a 1 mm Al filter. Scanning was performed in a high-resolution scanning mode for 4 minutes. Three-dimensional reconstruction was performed by using scanning data, and microstructure parameters of target bone tissues were analyzed through system analysis software, including the measurement of a bone mineral density (BMD), a bone volume fraction (BV/TV), a trabecular thickness (Tb.Th), a trabecular number (Tb.N), and trabecular separation (Tb.Sp).

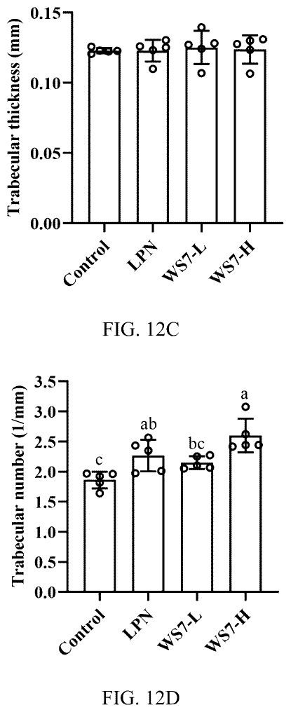

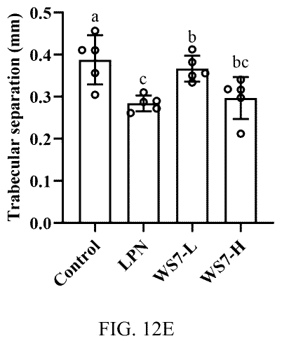

According to results shown in FIG. 11 and FIG. 12A˜ FIG. 12E, LPN and WS7-H interventions significantly improved a femoral microstructure: the BMDs of the two groups increased by 21.11% and 28.78% than that of the control group respectively, and the bone volume fractions thereof increased by 20.41% and 49.62% than that of the control group respectively (p<0.05), where the bone volume fraction of the WS7-H group was significantly higher than that of the LPN group (p<0.05); additionally, both the interventions increased the trabecular number (+21.76% and 39.70%, p<0.05) and decreased the trabecular separation (−26.66% and 23.44%, p<0.05); and three-dimensional reconstruction results also showed a decrease in an internal cavity volume of femur. The WS7-L group was significantly superior to the control group in trabecular separation (p<0.05). Additionally, there were no statistical differences in the trabecular thickness between the groups (p>0.05). Changes in the femoral microstructure are highly correlated with an increase in the bone strength, and the above results indicate that high-dose WS7 completely reproduces the improvement effect of LPN on a bone microstructure, and enhances the femoral strength by strengthening network connectivity of trabecular bones.

In summary, the results indicate that intragastric administration of WS7 at 0.41-4.1 mg/kg achieves almost the same effect as intragastric administration of LPN at 15 mg/kg in promoting bone growth performance, and WS7 demonstrates better safety, absorption efficiency, and bioavailability.

### Example 4: Preparation of a Polypeptide Gel Candy

The following raw materials in parts by weight are included: 4 kg of gelatin, 1 kg of starch, 1 kg of oligosaccharides, 10 kg of edible glycerol, 6 kg of water, 0.5 kg of WLKPDPS or NE(pS)PEQTDDL polypeptide dry powder obtained in Example 1, 0.3 kg of fruit flavor, 0.5 kg of phospholipid, and 7 kg of sunflower seed oil;
(1) all the raw materials were weighed according to the above weights;
(2) gelatin, starch, oligosaccharides, edible glycerol, and water were added to a mixing barrel, stirred for 30 minutes, and decocted to obtain a gel solution;
(3) polypeptide dry powder, fruit flavor, phospholipid, and sunflower seed oil were added to the mixing barrel, and stirred for 30 minutes to obtain a filling solution;
(4) the gel solution and the filling solution were molded through a candy machine, and cooled at 25° C. for 60 minutes to obtain a polypeptide gel candy.

Although the present disclosure has been disclosed in preferred examples, but they are not intended to limit the present disclosure. Anyone skilled in the art can make various changes and modifications without departing from the spirit and scope of the present disclosure. Therefore, the scope of protection of the present disclosure should be defined by the claims.

What is claimed is:

1. A milk-derived bioactive peptide, wherein an amino acid sequence of the milk-derived bioactive peptide is WLKPDPS or NESPEQTDDL.

2. The milk-derived bioactive peptide according to claim 1, wherein serine in the milk-derived bioactive peptide NESPEQTDDL is phosphorylated serine.

3. The milk-derived bioactive peptide according to claim 1, wherein a method for preparing the milk-derived bioactive peptide comprises a solid-phase synthesis method, an enzymatic hydrolysis method, and a microbial expression method.

4. A food, drug, health product, or nutritional product, wherein the food, drug, health product, or nutritional product contains an effective dose of the milk-derived bioactive peptide according to claim 1.

5. The food, drug, health product, or nutritional product according to claim 4, wherein the food, drug, health product, or nutritional product further contains the milk-derived bioactive peptide; a derivative of the milk-derived bioactive peptide refers to a milk-derived bioactive peptide derivative obtained by performing hydroxylation, carbonylation, carboxylation, methylation, acetylation, phosphorylation, esterification, or glycosylation modification on an amino acid side chain group, amino terminus, or carbonyl terminus of the milk-derived bioactive peptide.

6. The food, drug, health product, or nutritional product according to claim 4, wherein the drug further contains a pharmaceutically acceptable excipient; and the pharmaceutically acceptable excipient refers to a conventional pharmaceutical carrier in the pharmaceutical field.

7. The food, drug, health product, or nutritional product according to claim 6, wherein the excipient comprises one or more of the following: a binder which comprises a cellulose derivative, alginate, gelatin, or polyvinylpyrrolidone; a diluent which comprises starch, pregelatinized starch, dextrin, sucrose, lactose, or mannitol; a filler which comprises starch or sucrose; a humectant which comprises glycerol; a disintegrant which comprises sodium carboxymethyl starch, cross-linked polyvinylpyrrolidone, or dry starch; an absorption enhancer which comprises a quaternary ammonium compound; a surfactant which comprises polysorbate, sorbitan fatty acid ester, or fatty acid glyceride; a colorant which comprises titanium dioxide, sunset yellow, methylene blue, or pharmaceutical-grade iron oxide red; a lubricant which comprises hydrogenated vegetable oil, talc, or polyethylene glycol; a coating material which comprises acrylic resin, hydroxypropyl methylcellulose, povidone, or cellulose acetate phthalate; and any other adjuvant which comprises a flavoring agent or sweetener added to a composition.

8. The food, drug, health product, or nutritional product according to claim 4, wherein a dosage form of the drug comprises an oral dosage form, an injectable dosage form, and an inhalation dosage form; and the oral dosage form comprises but is not limited to a tablet, a capsule, a granule, an oral liquid, and an oral suspension.

9. The food, drug, health product, or nutritional product according to claim 4, wherein the injectable dosage form comprises an injection and an injection powder.

10. The food, drug, health product, or nutritional product according to claim 4, wherein the inhalation dosage form comprises an aerosol and a dry powder inhaler.

11. The food, drug, health product, or nutritional product according to claim 4, wherein the food comprises a cereal product, a vegetable product, a fruit product, a meat product, a seafood, an egg product, a dairy product, a soybean product, and a beverage;
the food further comprises a special dietary food; and
the health product further contains an acceptable excipient.

12. An application of the milk-derived bioactive peptide according to claim 1 in the preparation of a food, drug, health product, or nutritional product, wherein the food, drug, health product, or nutritional product is used for promoting calcium absorption.

13. The application according to claim 12, wherein the drug further contains a pharmaceutically acceptable excipient; and the pharmaceutically acceptable excipient refers to a conventional pharmaceutical carrier in the pharmaceutical field.

14. The application according to claim 13, wherein the excipient includes one or more of the following: a binder which comprises a cellulose derivative, alginate, gelatin, or polyvinylpyrrolidone; a diluent which comprises starch, pregelatinized starch, dextrin, sucrose, lactose, or mannitol; a filler which comprises starch or sucrose; a humectant which comprises glycerol; a disintegrant which comprises sodium carboxymethyl starch, cross-linked polyvinylpyrrolidone, or dry starch; an absorption enhancer which comprises a quaternary ammonium compound; a surfactant which comprises polysorbate, sorbitan fatty acid ester, or fatty acid glyceride; a colorant which comprises titanium dioxide, sunset yellow, methylene blue, or pharmaceutical-grade iron oxide red; a lubricant which comprises hydrogenated vegetable oil, talc, or polyethylene glycol; a coating material which comprises acrylic resin, hydroxypropyl methylcellulose, povidone, or cellulose acetate phthalate; and any other adjuvant which comprises a flavoring agent or sweetener added to a composition.

15. The application according to claim 12, wherein a dosage form of the drug comprises an oral dosage form, an injectable dosage form, and an inhalation dosage form.

16. The application according to claim 12, wherein the oral dosage form comprises a tablet, a capsule, a granule, an oral liquid, and an oral suspension.

17. The application according to claim 12, wherein the injectable dosage form comprises an injection and an injection powder.

18. The application according to claim 12, wherein the inhalation dosage form comprises an aerosol and a dry powder inhaler.
